(19) United States
(12) Patent Application Publication
(10) Pub. No.: US 2020/0237657 A1
(43) Pub. Date: Jul. 30, 2020

(54) NASAL MOISTURIZER

(71) Applicant: Matrixx Initiatives, Inc., Scottsdale, AZ (US)

(72) Inventors: Timothy L. Clarot, Phoenix, AZ (US); Arlene M. Ascarate, New River, AZ (US)

(73) Assignee: Matrixx Initiatives, Inc., Scottsdale, AZ (US)

(21) Appl. No.: 16/259,512

(22) Filed: Jan. 28, 2019

(51) Int. Cl. A61K 9/00; A61K 31/728; A61K 36/886; A61K 31/722

(52) U.S. Cl. CPC A61K 9/0043; A61K 31/728; A61K 31/723; A61K 31/722; A61K 36/886

(57) ABSTRACT

The present disclosure describes pharmaceutical compositions and methods for cleansing the nose, clearing the nose, imparting a cooling sensation in the nose, moisturizing nasal passages, soothing a dry nose, and ameliorating dehydration of the anterior nasal mucosa in humans in need of relief thereof. The compositions herein comprise humectants such as Aloe barbadensis leaf gel, hyaluronic acid, and/or a hyaluronate salt in a buffered and thickened pharmaceutically acceptable carrier. In various aspects, methods for relief comprise nasally administering compositions comprising Aloe barbadensis leaf gel and sodium hyaluronate in a pharmaceutically acceptable carrier to a human in need of relief thereof.

### FIELD OF THE INVENTION

The present disclosure relates generally to pharmaceutical compositions and methods of use thereof, and in particular to nasal compositions comprising humectants and methods to moisturize and cleanse the nasal passages in a human in need thereof.

### BACKGROUND OF THE INVENTION

A dry nose is a common problem in humans everywhere, particularly those residing in dry climates such as the Southwestern United States and the Middle East. A dry nose is irritating and painful at the very least but can also be accompanied by dehydration of the anterior mucosa, which leads quite often to bleeding. The dried blood adds to the debris that may need washing out.

Also, many nasal spray decongestants and antihistamines dry out the nasal passages. The increased air movement through opened sinuses also contributes to drying of mucosa from the increase in volume of dry air moving through the sinuses. Further, many prescription drugs lead to dry eyes and dry nasal mucosa.

Therefore, new nasal products specially formulated for moisturizing nasal passages and cleansing the nose are needed. For example, new pharmaceutical compositions usable for cleansing the nose, moisturizing nasal passageways, soothing a dry nose, and ameliorating dehydration of the anterior nasal mucosa are desirable.

### SUMMARY OF THE INVENTION

Various embodiments of the present disclosure provide compositions and methods for cleansing the nose, clearing the nose, moisturizing nasal passages, soothing a dry nose, and ameliorating dehydration of the anterior nasal mucosa.

In various embodiments, compositions that cleanse the nose comprise at least one humectant in a pharmaceutically acceptable carrier. In various embodiments, the at least one humectant is selected from the group consisting of Aloe barbadensis leaf gel, hyaluronic acid, hyaluronate salts, and mixtures thereof.

In various embodiments, compositions that clear the nose comprise at least one humectant in a pharmaceutically acceptable carrier. In various embodiments, the at least one humectant is selected from the group consisting of Aloe barbadensis leaf gel, hyaluronic acid, hyaluronate salts, and mixtures thereof.

In various embodiments, compositions that moisturize nasal passages comprise at least one humectant in a pharmaceutically acceptable carrier. In various embodiments, the at least one humectant is selected from the group consisting of Aloe barbadensis leaf gel, hyaluronic acid, hyaluronate salts, and mixtures thereof.

In various embodiments, compositions that soothe a dry nose comprise at least one humectant in a pharmaceutically acceptable carrier. In various embodiments, the at least one humectant is selected from the group consisting of Aloe barbadensis leaf gel, hyaluronic acid, hyaluronate salts, and mixtures thereof.

In various embodiments, compositions that ameliorate dehydration of the anterior nasal mucosa comprise at least one humectant in a pharmaceutically acceptable carrier. In various embodiments, the at least one humectant is selected from the group consisting of Aloe barbadensis leaf gel, hyaluronic acid, hyaluronate salts, and mixtures thereof.

In various embodiments, compositions in accordance with the present disclosure, useful for cleansing the nose, clearing the nose, moisturizing nasal passages, soothing a dry nose and ameliorating dehydration of the anterior nasal mucosa, comprise: (a) from about 0.0002 wt. % to about 0.02 wt. % of a mixture of Aloe barbadensis leaf gel and a hyaluronate salt; and (b) a pharmaceutically acceptable carrier. In various embodiments, the hyaluronate salt comprises sodium hyaluronate. In various aspects, the sodium hyaluronate has a molecular weight of from about 1,000,000 to about 1,500,000 Daltons.

In various embodiments, compositions in accordance with the present disclosure, useful for cleansing the nose, clearing the nose, moisturizing nasal passages, soothing a dry nose and ameliorating dehydration of the anterior nasal mucosa, comprise: (a) from about 0.0001 wt. % to about 0.01 wt. % Aloe barbadensis leaf gel; (b) from about 0.0001 wt. % to about 0.01 wt. % of a hyaluronate salt; and (c) a pharmaceutically acceptable carrier. In various embodiments, the hyaluronate salt comprises sodium hyaluronate. In various aspects, the sodium hyaluronate salt has a molecular weight of from about 1,000,000 to about 1,500,000 Daltons.

In various embodiments, compositions in accordance with the present disclosure, useful for cleansing the nose, clearing the nose, moisturizing nasal passages, soothing a dry nose and ameliorating dehydration of the anterior nasal mucosa, comprise: (a) at least one or a combination of: Aloe barbadensis leaf gel at from about 0.0001 wt. % to about 0.01 wt. %; and a hyaluronate salt at from about 0.0001 wt. % to about 0.01 wt. %; (b) a thickener at about 0.001 wt. % to about 10 wt. %; (c) a surfactant at about 0.001 wt. % to about 10 wt. %; (d) an organic acid at about 0.01 wt. % to about 10 wt. %; (e) at least one of a carbonate, sulfate, and phosphate totaling about 0.01 wt. % to about 10 wt. %; (f) at least one inhalant at a total of about 0.001 wt. % to about 1 wt. %; (g) optionally at least one amino acid at a total of about 0.01 wt. % to about 10 wt. %; (h) optionally at least one salt at a total of about 0.01 wt. % to about 1 wt. %; (i) optionally at least one fragrance at a total of about 0.001 wt. % to about 1 wt. %; (j) optionally at least one sweetener at a total of about 0.001 wt. % to about 1 wt. %; (k) optionally at least one of a preservative, ultraviolet inhibitor and antioxidant at a total of about 0.001 wt. % to about 1 wt. %; (l) optionally a non-aqueous solvent or vehicle at about 0.001 wt. % to about 99 wt. %; and (m) remainder water, wherein each wt. % is based on the total weight of the composition.

In various embodiments, a method for cleansing the nose, clearing the nose, moisturizing nasal passages, soothing a dry nose, or ameliorating dehydration of the anterior nasal mucosa, comprises applying to the nasal passageways of a human in need thereof a therapeutically effective amount of a composition comprising Aloe barbadensis leaf gel and a hyaluronate salt in a pharmaceutically acceptable carrier. In various embodiments, the pharmaceutically acceptable carrier is predominately water. In various embodiments, the composition further comprises any combinations of thickener, surfactant, acid, alkaline material, inhalant, amino acid, salt, drug active, fragrance, sweetener, and/or preservative.

In various embodiments, a method for cleansing the nose, clearing the nose, moisturizing nasal passages, soothing a dry nose, or ameliorating dehydration of the anterior nasal mucosa, comprises nasal administration of a therapeutically effective amount of a pharmaceutical composition comprising Aloe barbadensis leaf gel and a hyaluronate salt in a pharmaceutically acceptable carrier. In various embodiments, the pharmaceutically acceptable carrier is predominately water. In various embodiments, the composition further comprises any combination of thickener, surfactant, acid, alkaline material, inhalant, amino acid, salt, drug active, fragrance, sweetener, and/or preservative. In certain aspects, the hyaluronate salt comprises sodium hyaluronate.

In various embodiments, a method for cleansing the nose, clearing the nose, moisturizing nasal passages, soothing a dry nose, or ameliorating dehydration of the anterior nasal mucosa in a human comprises nasally administering a therapeutically effective amount of a pharmaceutical composition consisting essentially of: (a) from about 0.0002 wt. % to about 0.02 wt. % of a mixture of Aloe barbadensis leaf gel and a hyaluronate salt; (b) from about 0.5 wt. % to about 1.5 wt. % of a cellulosic thickener; (c) from about 0.01 wt. % to about 1 wt. % of a surfactant; (d) from about 0.1 wt. % to about 1 wt. % of an organic acid; (e) from about 0.1 to about 2 wt. % of a mixture of sodium monobasic and sodium dibasic phosphate salts; (f) from about 0.05 wt. % to about 1 wt. % of a mixture of menthol, eucalyptol and eugenol; (g) from about 0.05 wt. % to about 0.5 wt. % of an amino acid; (h) from about 0.1 wt. % to about 1 wt. % sodium chloride; and (i) from about 90 wt. % to about 99 wt. % water, wherein each wt. % is based on the total weight of the composition. In various aspects, the hyaluronate salt comprises sodium hyaluronate.

In various embodiments, compositions useful for cleansing the nose, clearing the nose, moisturizing nasal passages, soothing a dry nose and ameliorating dehydration of the anterior nasal mucosa, are formulated into physical forms, such as, for example, liquid sprays and prewetted swabs, amenable to administration to the nasal mucosa of the subject in need of relief thereof. In various embodiments, administration of such compositions comprises applying a therapeutically effective amount of the composition into one or both nostrils of the human in need of relief thereof, such as by spraying the composition or by inserted a swab prewetted with the composition into one of both nostrils.

### DETAILED DESCRIPTION OF THE INVENTION

The following description is of various exemplary embodiments only, and is not intended to limit the scope, applicability or configuration of the present disclosure in any way. Rather, the following description is intended to provide a convenient illustration for implementing various embodiments including the best mode. As will become apparent, various changes may be made in the function and arrangement of the elements described in these embodiments without departing from principles of the present disclosure.

As described in more detail herein, various embodiments of the present disclosure generally comprise a composition useful for cleansing the nose, clearing the nose, moisturizing nasal passages, soothing a dry nose, and ameliorating dehydration of the anterior nasal mucosa, wherein the composition comprises: at least one humectant; and a pharmaceutically acceptable carrier.

As described in more detail herein, various embodiments of the present disclosure generally comprise a method for cleansing the nose, clearing the nose, moisturizing nasal passages, soothing a dry nose, or ameliorating dehydration of the anterior nasal mucosa, the method comprising nasally administering to a human in need of relief thereof a therapeutically effective amount of a composition comprising at least one humectant; and a pharmaceutically acceptable carrier. The composition may be in the form of a sprayable liquid or the composition may be disposed on the absorbent end of a swab.

The Humectant

As used herein, the term “humectant” takes on its ordinary meaning in formulation chemistry of a substance that facilitates moisture absorption and/or retention in tissue. Generally, a humectant is hygroscopic such that the substance attracts and holds water molecules through some degree of hydrogen bonding. Thus, when a humectant is present in the derma or mucosa, such as by using a skin cream or a nasal moisturizer including the humectant, the hygroscopic humectant will attract water from the surroundings and provide moisturizing of the derma or mucosa.

A wide range of chemical substances can function as humectants, and quite often the commonality between the humectants is only the hygroscopic properties rather than a common chemical structure, although the hygroscopicity is intrinsically available to molecules having sufficient hydroxyl (—OH), carboxylate (—CO₂H) and/or amine (—NH₂) substituents that can hydrogen bond water molecules. Some humectants, such as polysaccharides, have a great number of —OH groups, such as if carbohydrate in nature and following the generic structure of Cₓ(H₂O)ₓ.

In various embodiments, any humectant disclosed in McCutcheon's, “Functional Materials Vol. 2: North America,” First Edition 1991, (ISBN: 0944254101), falls within the scope of the present disclosure and finds use in the nasal cleansing and moisturizing compositions herein.

For use herein, a humectant comprises a chemical substance having at least one hydroxyl (—OH), carboxylate (—CO₂H) or amine (—NH₂) substituent. These humectants may be, for example, alcohols, glycols, polysaccharides, sugar polyols, or vitamins, and may include such natural materials as Aloe barbadensis (Aloe Vera) gel, honey, glycerin and D-panthenol.

In various embodiments, the compositions in accordance to the present disclosure comprise at least one humectant, present in a total of amount of from about 0.0002 to about 0.02 wt. %, based on the total weight of the composition. In certain aspects, the compositions comprise a mixture of Aloe barbadensis leaf gel at from about 0.0001 to about 0.01 wt. % and hyaluronic acid or a corresponding hyaluronate salt at from about 0.0001 to about 0.01 wt. %. Each of the various humectants for use herein are discussed below.

Aloe Barbadensis Leaf Gel

Aloe barbadensis leaf gel is the natural inner clear gel material from the fleshy leaves of the Aloe barbadensis miller plant. The plant is often referred to as Aloe barbadensis or simply as Aloe Vera. The liquid material contains at least 90% by weight water, the remainder being a complex mixture of glucomannans, amino acids, lipids, sterols, enzymes, minerals, anthraquinones, fatty acids, hormones, and vitamins. As used herein, Aloe barbadensis leaf gel may be a combination of materials from the mucilage layer of the plant, known to be rich in humectant materials such as monosaccharides, glucomannans, polymannose and the mucopolysaccharides. For a review of Aloe Vera see, for example, A. Surjushe, et al., “Aloe Vera: A Short Review,” Indian J. Dermatol., 53(4), 163-166, 2008. Aloe barbadensis leaf gel for use herein may be sourced from Aromatics International, Florence, Mont. Their organic Aloe Vera Gel is obtained by cold pressing the whole leaf of mature Aloe Vera plants.

Aloe barbadensis leaf gel may be incorporated in the compositions of the present disclosure at levels of about 0.0001 wt. % to about 0.01 wt. %, based on the total weight of the composition. In certain examples, Aloe barbadensis leaf gel may be present in the compositions of the present disclosure at levels of about 0.001 wt. %, based on the total weight of the composition.

Hyaluronic Acid and Corresponding Hyaluronate Salts

Hyaluronic acid is a polysaccharide, and more specifically an anionic non-sulfated glycosaminoglycan, having the molecular formula (C₁₄H₂₁NO₁₁)n, wherein n is on the order of 10's of thousands. The repeating monomer is a disaccharide composed of D-glucuronic acid and N-acetyl-D-glucosamine, linked by alternating β-(1→4) and β-(1→3) glycosidic bonds. The substance naturally occurs in animals and has a molecular weight up to about 7-million Daltons. Although the substance is hygroscopic and functions as a humectant, it is also known to provide a mechanical barrier in wound healing. Thus, in the present nasal compositions, hyaluronic acid or its corresponding salts can act as barriers and protectants to the nasal mucosa, in addition to a hydrating agent (i.e. humectant). Since hyaluronic acid comprises one carboxylic acid moiety (—CO₂H) per monomeric disaccharide unit (on the D-glucuronic acid portion of the disaccharide), hyaluronic acid can exist as a salt (i.e., a hyaluronate salt, a conjugate base with an associated cation) by deprotonation of these carboxylic acid groups. Hyaluronate salts may be partial or full salts (i.e., partially or fully neutralized with respect to the all of the 10's of thousands of —CO₂H substituents), or mixed salts, comprising any number and combination of cations such as Na⁺, K⁺, Mg²⁺, Ca²⁺, and so forth. The sodium salt is known and is soluble in water. Sodium hyaluronate is also naturally occurring and is believed to be a tissue lubricant in vivo. Sodium hyaluronate for use in the present compositions has a molecular weight of about 1.0×10⁶ to about 1.5×10⁶ Daltons and may be sourced, for example, from Charkit Chemical Co., Norwalk, Conn., or other suppliers.

Hyaluronic acid, or a hyaluronate salt, may be incorporated in the compositions of the present disclosure at levels of about 0.0001 wt. % to about 0.01 wt. %, based on the total weight of the composition. In certain embodiments, a nasal composition in accordance with the present disclosure comprises about 0.001 wt. % sodium hyaluronate having a molecular weight of about 1.0×10⁶ to about 1.5×10⁶ Daltons. It should be understood that when sodium hyaluronate is formulated into a pharmaceutical composition at a particular pH, such as a composition buffered to pH less than about 6, some of the sodium hyaluronate may exist as a partial salt or as hyaluronic acid. In other words, once formulated, the hyaluronate may exist as an equilibrium mixture of a multitude of partial salts, the full salt and hyaluronic acid. For purposes herein, the starting material used to formulate the compositions herein comprises sodium hyaluronate, and it is not practical or even necessary to ascertain how many or which —CO₂⁻Na⁺ groups may be protonated back to —CO₂H groups because of the pH of the composition.

In various embodiments, compositions of the present disclosure may comprise a mixture of Aloe barbadensis leaf gel and a hyaluronate salt at a total level of about 0.0002 wt. % to about 0.02 wt. %, based on the total weight of the composition.

In various embodiments, compositions of the present disclosure comprise Aloe barbadensis leaf gel at from about 0.0001 wt. % to about 0.01 wt. %, based on the total weight of the composition, and sodium hyaluronate at from about 0.0001 wt. % to about 0.01 wt. %, based on the total weight of the composition.

The Pharmaceutically Acceptable Carrier

In various embodiments, a pharmaceutical acceptable carrier in accordance with the present disclosure may comprise any one or combinations of thickeners, surface modifying agents (surfactants), pH adjusters, pH buffers, amino acids, inhalants, fragrances, salts, drug actives, preservatives, uv inhibitors, antioxidants, flavorings, sweeteners, water, non-aqueous solvents, petroleum-based vehicles, and other adjuvants, such as to achieve a particular dosage form, final concentration(s) of humectant(s) and/or other substances, method of administration, therapeutic efficacy, cost, storage stability, consumer acceptability, marketing advantage, and/or any other technical or business goal.

Thickeners

Pharmaceutical compositions in accordance with various embodiments of the present disclosure may further comprise one or more thickeners. Such materials may be natural, synthetic or semisynthetic, and may be organic/polymeric or inorganic substances, or mixtures thereof. Polymers may include homo-polymers, random co-polymers and block co-polymers. Polymers may also include proteins such as albumin, or other natural polymers such as chitin or xanthan. Inorganic thickening agents may include, but are not limited to, such materials as clays and silica gel. A thickener used herein may be nonionic, anionic, cationic, or amphoteric, or an inorganic mineral or salt. A thickener may be incorporated as its salt (partial or full) having any combinations of positively or negatively charged counterion(s), (e.g., Na⁺, K⁺, Cl⁻, etc.) as appropriate for the particular ionizable groups of the thickener.

Thickeners may be used to provide any one, or combination of, bulk, viscosity or rheology characteristics in the compositions. In various embodiments, one or more thickeners may be added to impart certain rheology characteristics to the present compositions, such as a desired shear, yield, deformation, plasticity, elasticity, viscoelasticity, pseudo-plasticity, or the like. In various embodiments, one or more thickeners, acting as bulking agent(s), may also be added to reach a particular bulk density target. In various embodiments, one or more thickeners may also be added to impart other physical characteristics such as a particular spray droplet size, dispensing dose size, cling and/or feel within the nasal passages or in the mouth if/when the composition drains back.

Thickening agents include, but are not limited to, carboxymethyl cellulose, carboxyethyl cellulose, hydroxyethyl cellulose, hydrophobically modified hydroxyethyl cellulose, hydroxypropyl cellulose, hydroxypropyl methyl cellulose, methyl cellulose, ethyl cellulose, microcrystalline cellulose, nitrocellulose and other cellulosic thickeners, polyvinyl alcohol, polyvinylpyrrolidone, polyvinylmethacrylate, polyacrylates, acrylate co-polymers such as acrylic acid/vinyl pyrrolidone cross-polymer, carboxyvinyl polymers, polyvinylacetate, polyvinyl co-polymers, polyurethanes, various starches, modified starches, dextrin, xanthan and other gums, agar, alginic acid and alginates, pectin, gelatin and other hydrocolloids, gelling agents, casein, albumin, chitin, collagen, silica gel, fumed silica, magnesium aluminum silicates, clay, bentonite, hectorite, and combinations thereof. Various cellulosic thickeners, such as hydroxypropyl methyl cellulose (hypromellose), are available from Dupont/Dow under the tradename Methocel®.

One or more thickeners may be incorporated in the compositions of the present disclosure at levels of about 0.001 wt. % to about 10 wt. %, based on the total weight of the composition.

In various embodiments, the compositions of the present disclosure comprise a cellulosic thickener at a level of about 0.001 wt. % to about 10 wt. %, based on the total weight of the composition. In various embodiments, the compositions of the present disclosure comprise hydroxypropyl methyl cellulose at a level of about 0.001 wt. % to about 10 wt. %, based on the total weight of the composition.

In various embodiments, the compositions of the present disclosure have a viscosity of greater than about 1000 cps. In certain examples, the compositions of the present disclosure have a viscosity of less than about 500 cps. In various aspects, compositions of the present disclosure comprise about 1.0 wt. % of a cellulosic thickener, based on the total weight of the composition, and have a viscosity of less than about 500 cps.

Surfactants

Pharmaceutical compositions in accordance with various embodiments of the present disclosure may further comprise one or more surfactants. As used herein, the term “surfactant” is intended to include emulsifiers and solubilizers because some surfactants function as emulsifiers or solubilizers depending on their chemical structure and the nature of co-ingredients in a particular composition. Surfactants for use herein may be anionic, nonionic, cationic, or amphoteric.

Exemplary anionic surfactants include, but are not limited to, fatty acids, alkyl sulfates, alkyl ether sulfates, alkyl aryl sulfonates, alkyl succinates, alkyl sulfosuccinates, N-alkoyl sarcosinates, alkyl phosphates, alkyl ether phosphates, alkyl ether carboxylates, alkylamino acids, alkyl peptides, alkoyl taurates, acyl and alkyl glutamates, alkyl isethionates, α-olefin sulfonates, and combinations thereof.

Exemplary nonionic surfactants include, but are not limited to, aliphatic primary or secondary linear or branched chain fatty alcohols or phenols, fatty acid esters, mono-, di-, and tri-fatty acid glycerides, alkyl alkoxylates, alkyl phenol alkoxylates, block alkylene oxide condensates of alkyl phenols, alkylene oxide condensates of alkanols, ethylene oxide/propylene oxide (EO/PO) block copolymers, amine oxides, phosphine oxides, mono- or di-alkyl alkanolamides, alkyl polysaccharides, sorbitan fatty acid esters, polyoxyethylene sorbitan fatty acid esters, polyoxyethylene sorbitol esters, polyoxyethylene esters, polyoxyethylene alcohols, mono- and diethanolamides, polyglycosides, polyglucosides, diglucoside, alkyl polyglucoside, polysorbates, alkoxylated fatty alcohols, alkoxylated fatty acid glycerides, and mixtures thereof.

Exemplary amphoteric surfactants include, but are not limited to, alkyl betaines, alkyl amidopropyl betaines, alkyl sulfobetaines, alkyl glycinates, alkyl carboxyglycinates, alkyl amphopropionates, alkyl amidopropyl hydroxysultaines, acyl taurates, acyl glutamates, and mixtures thereof.

Exemplary cationic surfactants include, but are not limited to, quaternary alkyl amines, alkyl imidazolines, quaternary ethoxylated amines, quaternized amides, and combinations thereof. Some quaternary compounds, such as benzalkonium chloride, can function as antimicrobial agents (e.g. preservatives, discussed herein below), although they are cationic surfactants structurally.

One or more surfactants may be incorporated in the compositions of the present disclosure at levels of about 0.001 wt. % to about 10 wt. %, based on the total weight of the composition.

In various embodiments, the compositions of the present disclosure comprise the nonionic surfactant, polyethoxylated sorbitan laurate and/or oleate ester at a level of about 0.01 to about 1.0 wt. %, based on the total weight of the composition. In various embodiments, the compositions of the present disclosure comprise polyoxyethylene (20) sorbitan monooleate (polysorbate 80, or TWEEN® 80) at a level of about 0.01 to about 1.0 wt. %, based on the total weight of the composition.

pH Adjusters and pH Buffers

Pharmaceutical compositions in accordance with various embodiments of the present disclosure may further comprise one or more acidifying agents or alkaline agents as necessary to neutralize various co-ingredients, form salts of various co-ingredients, and/or achieve a particular pH target for the composition. In various embodiments of the present disclosure, the compositions are formulated to be acidic, i.e., having a pH of less than about 7. In various embodiments, the pH of the composition is from about 2 to about 6. In certain aspects, the pH of the composition is from about 3 to about 5. Combinations of various acidifying agents and alkaline agents may be used to create buffering systems that stabilize the desired final pH of the composition. Buffers may be mixed buffers, meaning that the alkaline agent is not necessarily the conjugate base of the acidifying agent.

Exemplary acidifying agents for use in the present compositions include, but are not limited to, organic acids of any molecular weight and mineral acids (inorganic acids), and mixtures thereof. Organic acids may include mono-carboxylic acids, di-carboxylic acids, or tri-carboxylic acids, and may be saturated or may have any degree of unsaturation. For example, organic acids for use in various embodiments of the composition in accordance to the present disclosure may include, but are not limited to, formic acid, carbonic acid, acetic acid, lactic acid, oxalic acid, propionic acid, valeric acid, enanthic acid, pelargonic acid, butyric acid, lauric acid, docosahexaenoic acid, eicosapentaenoic acid, pyruvic acid, acetoacetic acid, benzoic acid, salicylic acid, aldaric acid, fumaric acid, glutaconic acid, traumatic acid, muconic acid, malonic acid, malic acid, succinic acid, glutaric acid, adipic acid, pimelic acid, suberic acid, azelaic acid, abietic acid, pimaric acid, sebacic acid, phthalic acid, isophthalic acid, terephthalic acid, maleic acid, citric acid, and combinations thereof. For example, mineral acids for use in various embodiments of the composition in accordance to the present disclosure may include, but are not limited to hydrochloric acid, phosphoric acid, sulfuric acid, nitric acid, and combinations thereof.

One or more organic and/or mineral acids may be incorporated in the compositions of the present disclosure at levels of about 0.01 wt. % to about 10 wt. %, based on the total weight of the composition.

In various embodiments, the compositions of the present disclosure comprise at least one mono-carboxylic acid at a level of about 0.01 to about 10 wt. %, based on the total weight of the composition. In various embodiments, the compositions of the present disclosure comprise at least one di-carboxylic acid at a level of about 0.01 to about 10 wt. %, based on the total weight of the composition. In various embodiments, the compositions of the present disclosure comprise at least one tri-carboxylic acid at a level of about 0.01 to about 10 wt. %, based on the total weight of the composition. In various embodiments, the compositions of the present disclosure comprise any single one, or combination, of mono-, di-, and tri-carboxylic acids. In various embodiments, the compositions of the present disclosure comprise at least one of malonic, maleic, succinic, glutaric and adipic acids, at a total level of about 0.01 to about 10 wt. %, based on the total weight of the composition. In various embodiments, the compositions of the present disclosure comprise succinic acid at a level of about 0.01 to about 1 wt. %, based on the total weight of the composition.

Exemplary alkaline materials include any organic amines, NH₃, alkali metal or alkaline earth hydroxide, any conjugate bases of any organic acids (e.g. R—COO⁻), and any of the salts of carbonic acid, phosphoric acid, nitric acid and sulfuric acid, and any mixtures thereof. For example, alkaline materials for use in various embodiments of the composition in accordance to the present disclosure may include, but are not limited to, NaOH, KOH, NH₃, sodium acetate, sodium succinate, disodium succinate, monosodium citrate, disodium citrate, trisodium citrate, NaH₂PO₄, Na₂HPO₄, Na₃PO₄, KH₂PO₄, K₂HPO₄, K₃PO₄, NaHSO₄, Na₂SO₄, KHSO₄, K₂SO₄, NaHCO₃, Na₂CO₃, KHCO₃, K₂CO₃, NaH₃P₂O₇, Na₂H₂P₂O₇, Na₃HP₂O₇, Na₄P₂O₇, KH₃P₂O₇, K₂H₂P₂O₇, K₃HP₂O₇, K₄P₂O₇, and mixtures thereof. Any of these chemical species may exist as various hydrates when purchased as raw materials for use in the present compositions.

One or more alkaline agents may be incorporated in the compositions of the present disclosure at levels of about 0.01 wt. % to about 10 wt. %, based on the total weight of the composition.

In various embodiments, the compositions of the present disclosure comprise at least one salt of carbonic acid or phosphoric acid at a level of about 0.01 to about 10 wt. %, based on the total weight of the composition. In various embodiments, the compositions of the present disclosure comprise at least one of sodium phosphate dibasic (Na₂HPO₄) and sodium phosphate monobasic (NaH₂PO₄) at a level of about 0.1 to about 5 wt. %, based on the total weight of the composition. In various embodiments, the compositions of the present disclosure comprise sodium phosphate dibasic (Na₂HPO₄) and sodium phosphate monobasic (NaH₂PO₄) at a total level of about 0.1 to about 5 wt. %, based on the total weight of the composition.

In various embodiments, the compositions of the present disclosure comprise sodium phosphate dibasic (Na₂HPO₄) and sodium phosphate monobasic (NaH₂PO₄), at a total level of about 0.1 to about 5 wt. %, based on the total weight of the composition, and at least one of malonic, maleic, succinic, glutaric and adipic acids, at a level of about 0.01 to about 10 wt. %, based on the total weight of the composition, to form a final pH of about 2-6 and a mixed buffer system. In various embodiments, the compositions of the present disclosure comprise sodium phosphate dibasic (Na₂HPO₄) and sodium phosphate monobasic (NaH₂PO₄), at a total level of about 0.1 to about 5 wt. %, based on the total weight of the composition, and succinic acid at a level of about 0.01 to about 10 wt. %, based on the total weight of the composition, to form a final pH of about 3 to about 5, and a mixed buffer system.

Amino Acids

Pharmaceutical compositions in accordance with various embodiments of the present disclosure may further comprise one or more natural, synthetic, semi-synthetic, common, uncommon, known, or unknown amino acids, in any combination, wherein the one or more amino acids comprise any juxtaposition of the —NH₂ and —CO₂H substituents, (e.g., α-, β, γ, δ, etc.).

Table 1 lists common α-amino acids that, in various embodiments, find use in the compositions of the present disclosure. A number of these amino acids are known to possess antimicrobial efficacy. For example, L-lysine is a known antiviral agent and combinations of glycine with other amino acids have been shown to promote wound healing. Amino acids, in combination with the acidifying and alkaline agents above, may provide pH adjusting and pH buffering functions.

TABLE 1
Common α-Amino Acids

| Amino Acid | Abbreviation | Formula (molecular weight) |
|---|---|---|
| Alanine | Ala | C₃H₇NO₂ (89.09) |
| Arginine | Arg | C₆H₁₄N₄O₂ (174.20) |
| Asparagine | Asn | C₄H₈N₂O₃ (132.12) |
| Aspartic Acid | Asp | C₄H₇NO₄ (133.10) |
| Cysteine | Cys | C₃H₇NO₂S (240.30) |
| Glutamic Acid | Glu | C₅H₉NO₄ (147.13) |
| Glutamine | Gln | C₅H₁₀N₂O₃ (146.15) |
| Glycine | Gly | C₂H₅O₂ (75.07) |
| Histidine | His | C₆H₉N₃O₂ (155.16) |
| Isoleucine | Ile | C₆H₁₃NO₂ (131.18) |
| Leucine | Leu | C₆H₁₃NO₂ (131.18) |
| Lysine | Lys | C₆H₁₄N₂O₂ (146.19) |
| Methionine | Met | C₅H₁₁NO₂S (149.21) |
| Phenylalanine | Phe | C₉H₁₁NO₂ (165.19) |
| Proline | Pro | C₅H₉NO₂ (115.13) |
| Serine | Ser | C₃H₇NO₃ (105.19) |
| Threonine | Thr | C₄H₉NO₃ (119.12) |
| Tryptophan | Trp | C₁₁H₁₀N₂O₂ (204.23) |
| Tyrosine | Tyr | C₉H₁₁NO₃ (181.19) |
| Valine | Val | C₅H₁₁NO₂ (117.15) |

One or more amino acids may be incorporated in the compositions of the present disclosure at levels of about 0.01 wt. % to about 10 wt. %, based on the total weight of the composition.

In various embodiments, from about 0.01 wt. % to about 10 wt. % of at least one of glycine and L-lysine is used, based on the total weight of the composition. In various embodiments, the compositions of the present disclosure comprise from about 0.01 wt. % to about 10 wt. % glycine, based on the total weight of the composition.

Inhalants

The term “inhalant,” as used herein, refers to organic substances that have a notable “aromatic” or otherwise “strong” odor, sometimes associated with vaporizers and other home remedies to relieve sinus congestion. “Aromatic” is not strictly used herein to limit the inhalants to aromatic compounds in the organic chemistry sense (i.e., those compounds having aromatic unsaturation), but rather to include all molecules having a low vapor pressure and a smell that may be described, for example, as “medicinal.” Inhalants, as classified herein, overlap to some degree with fragrances. Inhalants may include aromatic compounds (i.e. compounds having an unsaturated ring), terpene or terpenoid compounds, small molecular weight volatile organic compounds, amongst others, and combinations thereof. Lists of useful inhalants for the compositions of the present disclosure may appear to be chemically unrelated. Inhalants finding use in the present disclosure may contain one or more chiral centers, in which case the particular inhalant(s) chosen for use may comprise a single enantiomer, a racemate, or any combination of diastereoisomers depending on the number of chiral centers and their individual chirality. Further, various oils may be used as well as inhalants when they contain volatile substances. Non-limiting examples include: Japanese peppermint oil, known to contain substantial amounts of menthol; orange oil, known to contain substantial amounts of D-limonene; and clove oil, known to contain a substantial amount of eugenol.

Exemplary inhalants for use in the present compositions include, but are not limited to, anethole, menthol, eucalyptol, borneol, borneol acetate, camphor, 1,8-cineole, cinnamaldehyde, benzaldehyde, citral, thujone, eugenol, limonene, geraniol, citronellol, citronellal, pinene, linalool, thymol, carvone, caryophyllene, linalyl acetate, methyl salicylate, and mixtures thereof. In various aspects, one or more of these inhalants provide a cooling sensation to the user.

One or more inhalants may be incorporated in the compositions of the present disclosure at levels of about 0.001 wt. % to about 1 wt. %, based on the total weight of the composition.

In various embodiments, the compositions of the present disclosure comprise at least one inhalant selected from the group consisting of eucalyptol, eugenol, menthol, camphor, and mixtures thereof, at a total level of from about 0.001 wt. % to about 1 wt. %, based on the total weight of the composition. In various embodiments, the compositions of the present disclosure comprise at least one of eucalyptol, eugenol, and menthol at a total level of about 0.001 wt. % to about 1 wt. %, based on the total weight of the composition. In various embodiments, the compositions of the present disclosure comprise eucalyptol, eugenol and menthol at a total level of about 0.001 wt. % to about 1 wt. %, based on the total weight of the composition.

Fragrances

In various embodiments, the compositions of the present disclosure may comprise at least one fragrance. As mentioned, fragrances overlap with odoriferous compounds mentioned above that, for purposes herein, were categorized as inhalants. Fragrances may comprise essential oils or may be synthetic or semi-synthetic. Some fragrances, for example various essential oils and phenolic solvents, have bacteriostatic and/or other preservative effects, and could equally be categorized and used as “preservatives” within the present disclosure.

Exemplary fragrances for use in the compositions of the present disclosure include, but are not limited to 3,3,5-trimethylcyclohexanol, methoxycyclohexanol, benzyl alcohol, anise alcohol, cinnamyl alcohol, β-phenyl ethyl alcohol (2-phenylethanol), cis-3-hexenol, musk xylol, isoeugenol, methyl eugenol, α-amylcinnamic aldehyde, anisaldehyde, n-butyl aldehyde, cumin aldehyde, cyclamen aldehyde, decanal, isobutyl aldehyde, hexyl aldehyde, heptyl aldehyde, n-nonyl aldehyde, nonadienol, hydroxycitronellal, benzaldehyde, methyl nonyl acetaldehyde, dodecanol, α-hexylcinnamic aldehyde, undecenal, heliotropin, vanillin, ethyl vanillin, methyl amyl ketone, methyl β-naphthyl ketone, methyl nonyl ketone, musk ketone, diacetyl, acetyl propionyl, acetyl butyryl, acetophenone, p-methyl acetophenone, ionone, methyl ionone, amyl butyrolactone, diphenyl oxide, methyl phenyl glycidate, γ-nonyl lactone, coumarin, cineole, ethyl methyl phenyl glycidate, methyl formate, isopropyl formate, linalyl formate, ethyl acetate, octyl acetate, methyl acetate, benzyl acetate, butyl propionate, isoamyl acetate, isopropyl isobutyrate, geranyl isovalerate, allyl capronate, butyl heptylate, octyl caprylate octyl, methyl heptynecarboxylate, methine octynecarboxylate, isoacyl caprylate, methyl laurate, ethyl myristate, methyl myristate, ethyl benzoate, benzyl benzoate, methylcarbinylphenyl acetate, isobutyl phenylacetate, methyl cinnamate, cinnamyl cinnamate, ethyl anisate, methyl anthranilate, ethyl pyruvate, ethyl α-butyl butylate, benzyl propionate, butyl acetate, butyl butyrate, p-tert-butylcyclohexyl acetate, cedryl acetate, citronellyl acetate, citronellyl formate, p-cresyl acetate, ethyl butyrate, ethyl caproate, ethyl cinnamate, ethyl phenylacetate, ethylene brassylate, geranyl acetate, geranyl formate, isoamyl salicylate, isoamyl isovalerate, isobornyl acetate, linalyl acetate, methyl anthranilate, methyl dihydrojasmonate, β-phenylethyl acetate, trichloromethylphenyl carbinyl acetate, terpinyl acetate, vetiveryl acetate, and mixtures thereof.

In various embodiments, the compositions of the present disclosure comprise at least one fragrance at levels of about 0.001 wt. % to about 1 wt. %, based on the total weight of the composition. In various embodiments, the compositions of the present disclosure comprise 2-phenylethanol at a level of about 0.001 wt. % to about 1 wt. %, based on the total weight of the composition.

Preservatives, uv Inhibitors and Antioxidants

In various embodiments, the compositions of the present disclosure may comprise at least one of a preservative, uv Inhibitor, and antioxidant. Preservatives, such as quaternary ammonium compounds, may be antimicrobial in function, for example, exhibiting protection against mold and bacteria growth in finished products. Ultraviolet (uv) inhibitors protect the composition from damage by light. Antioxidants, such as BHT (butylated hydroxytoluene), can be used to protect compositions from oxidation.

In various embodiments, the compositions of the present disclosure comprise at least one of a preservative, ultraviolet inhibitor and antioxidant, at a level of about 0.001 wt. % to about 1 wt. %, based on the total weight of the composition.

In various embodiments, the compositions of the present disclosure comprise at least one of an N-alkyl-N-benzyl-N,N-dimethyl quaternary ammonium salt (an ADBAC quat), a uv inhibitor and BHT, each at levels of about 0.001 wt. % to about 1 wt. %, based on the total weight of the composition. In various embodiments, the compositions of the present disclosure comprise benzalkonium chloride at a level of about 0.001 wt. % to about 1 wt. %, based on the total weight of the composition. Benzalkonium chloride for use herein may be obtained as a 50% active solution.

Sweeteners

In various embodiments, the compositions of the present disclosure comprise a sweetener, such as, for example, any sugar (fructose, glucose, sucrose) or sugar alcohol (sucralose, and xylitol), potassium acesulfame, aspartame, neotame, saccharin, stevia, and mixtures thereof. In various embodiments, the compositions of the present disclosure comprise at least one sweetener at a level of about 0.001 wt. % to about 1 wt. %, based on the total weight of the composition. In various embodiments, the compositions of the present disclosure comprise sodium saccharin at a level of from about 0.001 wt. % to about 1 wt. %, based on the total weight of the composition.

Non-Aqueous Solvents/Vehicles

In various embodiments, the compositions of the present disclosure comprise a non-aqueous solvent, such as, for example, any alcohol or an oily or waxy vehicle (petrolatum, stearyl alcohol, lanolin, yellow wax, polyethylene glycol ointment, and the like). In various embodiments, the compositions of the present disclosure comprise ethyl alcohol (ethanol) at a level of about 0.001 wt. % to about 99 wt. %, based on the total weight of the composition. In various embodiments, the compositions of the present disclosure comprise an oily or waxy vehicle at a level of about 0.001 wt. % to about 99 wt. %, based on the total weight of the composition. In some instances, a non-aqueous vehicle may be used to create a gel composition.

Salts

In various embodiments, the compositions of the present disclosure comprise a salt, such as, for example, sodium chloride and/or potassium chloride. In various embodiments, the compositions of the present disclosure comprise sodium chloride at a level of about 0.01 wt. % to about 1 wt. %, based on the total weight of the composition.

Drug Actives

In various embodiments, the compositions of the present disclosure comprise at least one drug active, such as a decongestant or antihistamine. The term “drug active,” as used herein, refers to a drug having at least some decongestant or antihistamine activity when administered topically or systemically in/on a human. Such drugs may have been the subject of an FDA or foreign drug approval, presently in the market or marketed in the past, or may be entirely experimental, unknown, newly discovered, or as yet, undiscovered. Such drugs may be synthetic organic compounds or natural products, or derivatives thereof.

Exemplary drug actives for use in the compositions of the present disclosure include, but are not limited to, diphenhydramine, pseudoephedrine, and loratadine. A drug active may be used in the compositions of the present disclosure at a level of about 0.00001 wt. % to about 1 wt. %, depending on the nature of the drug active and the finished dosage form of the composition.

Water

The compositions in accordance with the present disclosure may comprise a significantly large amount of water, for example, when the finished composition is in the form of a liquid. For example, water can make up most of the pharmaceutically acceptable carrier by weight. In other physical forms besides liquids, the compositions in accordance with the present invention may have much less water, such as in the case of an ointment, gel or paste. The water used herein may be from any source and may have been subjected to any purification process prior to use. For example, the water may be distilled or reverse osmosis water.

In various embodiments, the compositions of the present disclosure comprise at least about 50 wt. % water, based on the total weight of the composition. In various embodiments, the compositions of the present disclosure comprise at least about 85 wt. % water, based on the total weight of the composition. In various embodiments, the compositions of the present disclosure comprise at least about 90 wt. % water, based on the total weight of the composition. In various embodiments, the compositions of the present disclosure comprise at least about 95 wt. % water, based on the total weight of the composition. In various embodiments, the compositions of the present disclosure comprise at least about 98 wt. % water, based on the total weight of the composition. In various embodiments, the compositions of the present disclosure comprise at least about 99 wt. % water, based on the total weight of the composition. In various embodiments, the compositions of the present disclosure comprise from about 90 wt. % to about 99 wt. % water, based on the total weight of the composition.

Dosage Form and General Methods of Treatment

Compositions formulated in accordance with the present disclosure may be expressed in any dosage form, such as, but not limited to, liquids amenable to drops or spray or for saturating the end of an absorbent swab, aerosols, pre-treated applicators, waxes, pastes, salves, gels or ointments, amongst other forms. As such, water and non-aqueous solvent amounts may be adjusted accordingly (even to the extreme of one over the other) to accommodate formulation into these various dosage forms. “Pastes,” as the term is used here, also incorporates highly viscous, non-aqueous petrolatum-based pharmaceutical compositions.

Usage of various compositions in accordance to the present disclosure include, but are not limited to, (1) cleansing the nose; (2) clearing the nose; (3) moisturizing nasal passages; (4) soothing dry nose; and (5) ameliorating dehydration of the anterior mucosa, in a human exhibiting such symptoms and in need of relief. Compositions of the present disclosure may find additional uses, such as, relief from allergens, pollutants and other airborne irritants. Various aromas made possible by the inhalants disclosed herein may impart a mentally soothing aspect to the compositions, such as seen in aromatherapy, and/or a cooling sensation in the nostrils.

In various embodiments, the compositions of the present disclosure comprise nasal compositions having physical form amendable to application to the nasal passages and/or mucosa of a human in need of treatment. These forms include, but are not limited to, gels, treated swabs (e.g. swabs pre-wetted with a liquid, gel or paste), liquids, and pastes.

In various embodiments, the compositions of the present disclosure are thin liquids, generally aqueous-based, nasal sprays that can be aerosolized through a non-aerosol sprayer, (e.g. a squeeze bottle having a pin-hole spray exit, or a finger-actuated non-aerosol sprayer), or through a pressurized aerosol system. As meant herein, a “thin liquid” resembles water, and has a viscosity of less than about 500 cps, (i.e., no discernable viscosity to the naked eye).

In various embodiments, the compositions of the present disclosure are formulated as liquids intended for nasal irrigation.

Liquid spray nasal compositions in accordance with the present disclosure may be administered into the nasal passages of a human in need of relief by spraying approximately 50-250 μL of a liquid composition form of the composition from any dispenser into one or both nostrils. Administration may be as needed, daily, or may follow any other effective dosage schedule (every day, every other day, bi-weekly, weekly, etc.). Administration of nasal compositions disclosed herein into the nostrils of the human in need of relief thereof may be once per day, twice per day, three times per day, four times per day, five times per day, six times per day, or as many times per day as required to moisturize nasal passages, cool, soothe a dry nose, or ameliorate dehydration of the anterior mucosa.

For example, in various embodiments, 50-250 μL of a nasal composition in accordance with the present disclosure may be sprayed once or twice or more into each nostril, up to six times or more per day, as frequently as daily, for each day the person in need of relief exhibits symptoms of a dry, irritated or bleeding nose.

For example, a typical regimen comprises two sprays of a nasal composition in accordance with the present disclosure up each nostril at least once per day and up to about 10-times per day, each day as needed to cleanse the nose, clear the nose, moisturize nasal passages, soothe a dry nose, or ameliorate dehydration of the anterior mucosa.

In various embodiments, compositions in accordance with the present disclosure may be packaged into dispensing bottles and placed into a combination package with a pharmaceutical drug. For example, a moisturizing nasal spray composition, formulated in accordance with the present disclosure, may be provided in a small, (e.g. 15-20 mL) dispensing spray bottle, and that bottle placed into a carton alongside a bottle containing a pharmaceutical drug composition, such as a bottle of aspirin or TYLENOL® capsules. In this way, the human in need of relief can self-administer the analgesic for pain relief and use the nasal spray composition of the present disclosure for relief of the dry nose symptoms such as pain, burning, itching and irritation.

In various embodiments, combination packages may comprise a carton having cellophane view windows such that all products packaged therein can be seen by the consumer. Also, use instructions may be provided on the carton, and/or as a separate insert, instructing the consumer on the combined usage of the products within the carton.

Similar dosing amounts and regimens are conceivable using pre-treated swabs, having a liquid, gel or paste form of the composition on the tip of the swab. In various embodiments, a liquid composition having viscosity of less than about 500 cps may be absorbed into the absorbent end of a retail swab by the user or may be provided to the user in the form of a pre-treated swab so that the user does not need to perform the absorbing step of the administration.

In various embodiments, nasal cleansing and moisturizing compositions of the present disclosure are nasally administered via pre-treated swabs. It is important to recognize that a pre-treated swab may be wetted with a thin liquid, although in some instances it may be desirable to pre-treat a swab with a gel, paste or ointment form of the composition. Of course, the compositions of the present disclosure may be provided in any physical form such as liquid, gel, paste or ointment, and the consumer who is preparing to self-administer the product into their nose may take their own dry swab to dip in the liquid composition, or may dispense the gel, paste or ointment product form onto the tip of the dry swab, such as by squeezing product from a tube, before administering the product in the nose.

Swab systems for nasally administering pharmaceutical compositions are disclosed, for example, in U.S. Pat. Nos. 6,516,947; 7,597,901; and 8,133,502, the disclosures of which are incorporated herein by reference in their entireties. Although the '901 and '502 Patents disclose gelled composition on swabs for nasal administration, the systems may be adapted for use with thin liquid compositions. The '947 Patent discloses a closed plastic container that is snapped open to reveal a pre-treated swab. Swab packaging such as disclosed in the '947 patent is also amenable to use with thin liquids. Such swab packaging systems are not necessarily one-time use products. For example, once the package is snapped open and the pre-treated swab removed and used in one nostril, the swab may be dipped back into the reservoir portion of the packaging to be rewetted.

The loading of liquid onto the swab tip of a swab package system is dependent upon the size and absorptivity of the tip, the absorptivity being dependent on the material (e.g., cotton, polyester fibers, sponge, etc.) that the tip of the swab is made from. In various aspects, compositions herein are provided in a sealed swab system (such as disclosed in the above-referenced patents) at levels of from about 0.25 mL to about 10.0 mL per container. These amounts allow re-dipping of the swab back into the opened reservoir for at least a second wetting and application but may be adjusted if users perceive the amount in the reservoir to be wasteful and perhaps in excess of what is practically needed for a “single use” into both nostrils.

In various embodiments, the liquid fill into a reservoir of a sealed swab dispensing system is from about 0.5 mL to about 1.5 mL, or in some instances, about 1 mL per reservoir. For compositions having viscosity greater than about 500 centipoises, the swab can be reinserted into the reservoir and wiped around the inside perimeter to pick up more gel or paste form product rather than relying on capillary action to wet the swab, such as the case with thin liquid product forms.

Exemplary Compositions and Methods of Use

A range of compositions useful for cleansing the nose, clearing the nose, imparting a cooling sensation in the nostrils, moisturizing nasal passages, soothing a dry nose, and ameliorating dehydration of the anterior nasal mucosa, is provided in Table 2, demonstrating the scope of the present disclosure.

TABLE 2
General Nasal Relief Compositions

| Ingredients | Acceptable Wt. % Range |
|---|---|
| Humectant(s) | 0.0002-0.02 wt. % in total |
| Thickener | 0.001-10 wt. % |
| Surfactant | 0.001-10 wt. % |
| Organic Acid | 0.01-10 wt. % |
| Carbonate, sulfate and/or | 0.01-10 wt. % |
| phosphate materials | |
| Inhalant(s) | 0.001-1 wt. % in total |
| Amino acid (optional) | 0 wt. % or 0.01-1 wt. % |
| Salt (optional) | 0 wt. % or 0.01-1 wt. % |
| Fragrance (optional) | 0 wt. % or 0.001-1 wt. % |
| Sweetener (optional) | 0 wt. % or 0.001-1 wt. % |
| Preservative, uv inhibitor, | 0 wt. % or 0.001-1 wt. % |
| and/or antioxidant (optional) | |
| Non-aqueous solvent/vehicle | 0 wt. % or 0.001-99 wt. % |
| (optional) | |
| Water | Remainder |

A specific example of a composition useful for cleansing the nose, clearing the nose, imparting a cooling sensation in the nostrils, moisturizing nasal passages, soothing a dry nose, or ameliorating dehydration of the anterior nasal mucosa is set forth in Table 3. NASAL RELIEF COMPOSITION #1 in Table 3 had pH of about 3.5 to 4.8, and viscosity of less than 500 cps (spindle #3, 50 rpm, 25° C.). The composition was observably transparent and colorless (i.e., “water white”). The composition of Table 3 was packaged into sealed swab dispensing packaging at a level of 1 mL per package for use in the In-Home Use Test (IHUT) detailed below.

TABLE 3
NASAL RELIEF COMPOSITION #1

| Ingredients | CAS No. | Wt. % |
|---|---|---|
| Humectants | | |
| Aloe barbadensis leaf gel | 94349-62-9 | 0.001 |
| Sodium hyaluronate | 9067-32-7 | 0.001 |
| (MW = 1 × 10⁶ to 1.5 × 10⁶ Daltons) | | |
| Pharmaceutically | | |
| acceptable carrier | | |
| Hypromellose | 9004-65-3 | 1.000 |
| (Methocel ® E4) | | |
| Polysorbate 80 | 9005-65-6 | 0.080 |
| Succinic acid | 110-15-6 | 0.385 |
| NaH₂PO₄ | 10049-21-5 | 0.540 |
| Na₂HPO₄ | 7782-85-6 | 0.650 |
| Glycine | 56-40-6 | 0.100 |
| Eucalyptol | 470-82-6 | 0.025 |
| (Inhalant mixture component) | | |
| Eugenol | 97-53-0 | 0.002 |
| (Inhalant mixture component) | | |
| Menthol, crystalline | 2216-51-5 | 0.060 |
| (inhalant mixture component) | | |
| Fragrance | 60-12-8 | 0.020 |
| (2-phenylethanol) | | |
| Sodium chloride | 7647-14-5 | 0.450 |
| Sweetener | 6155-57-3 | 0.030 |
| (sodium saccharin) | | |
| Preservative | 63449-41-2 | 0.020 |
| (benzalkonium chloride) | | |
| Water, purified | 7732-18-5 | 96.636 |
| TOTAL | | 100.000 |

The pharmaceutical compositions in Tables 2 and 3, along with similar liquid or gel compositions formulated in accordance with the present disclosure, e.g., having combinations of these classes of ingredients, are typically batch-prepared in a kettle with addition of ingredients as appropriate and simple mixing until uniform. The compositions disclosed herein can be thickened more or less to any degree as appropriate for a desired finished dosage form or to accommodate a particular dispenser or dosing regimen. In various embodiments, the compositions of Table 2 and Table 3 may be filled directly into small squeezable spray bottles or directly filled into and sealed within snap-open swab dispensing systems such as those disclosed in the above-referenced patents.

In various aspects, a method of using a liquid nasal cleansing and moisturizing composition sealed inside a pre-treated swab dispensing system that includes a pre-treated swab inside a sealed reservoir tube comprises holding a portion of the tube; snapping open the tube; pulling out the pre-treated swab; and nasally administering the composition into a first nostril. The method may further comprise reinserting the swab into the reservoir tube to re-saturate the swab tip with additional liquid composition; and nasally administering the composition to a second nostril. In various aspects, the liquid composition has a viscosity of less than about 500 cps. In other examples, the composition comprises a gel having a viscosity of at least 1,000 cps.

In various embodiments, a composition in accordance with the present disclosure, useful for cleansing the nose, clearing the nose, imparting a cooling sensation in the nostrils, moisturizing nasal passages, soothing a dry nose, or ameliorating dehydration of the anterior nasal mucosa, comprises: (a) at least one humectant; and (b) a pharmaceutically acceptable carrier.

In various embodiments, a composition in accordance with the present disclosure, useful for cleansing the nose, clearing the nose, imparting a cooling sensation in the nostrils, moisturizing nasal passages, soothing a dry nose, or ameliorating dehydration of the anterior nasal mucosa, comprises: (a) at least one humectant selected from the group consisting of Aloe barbadensis leaf gel, hyaluronic acid, a hyaluronate salt, and mixtures thereof; and (b) a pharmaceutically acceptable carrier.

In various embodiments, a composition in accordance with the present disclosure, useful for cleansing the nose, clearing the nose, imparting a cooling sensation in the nostrils, moisturizing nasal passages, soothing a dry nose, or ameliorating dehydration of the anterior nasal mucosa, comprises: (a) at least one humectant selected from the group consisting of Aloe barbadensis leaf gel, hyaluronic acid, a hyaluronate salt, and mixtures thereof, the total humectant present at from about 00002 wt. % and 0.02 wt. %; and (b) the remainder, a pharmaceutically acceptable carrier, wherein each wt. % is based on the total weight of the composition.

In various embodiments, a composition in accordance with the present disclosure, useful for cleansing the nose, clearing the nose, imparting a cooling sensation in the nostrils, moisturizing nasal passages, soothing a dry nose, or ameliorating dehydration of the anterior nasal mucosa, comprises: (a) Aloe barbadensis leaf gel at about 0.0001 wt. % to about 0.01 wt. %, and sodium hyaluronate at about 0.0001 wt. % to about 0.01 wt. %; and (b) a pharmaceutically acceptable carrier, wherein each wt. % is based on the total weight of the composition. In various aspects, the MW of the sodium hyaluronate is about 1×10⁶ to about 1.5×10⁶ Daltons.

In various embodiments, a composition in accordance with the present disclosure, useful for cleansing the nose, clearing the nose, imparting a cooling sensation in the nostrils, moisturizing nasal passages, soothing a dry nose, or ameliorating dehydration of the anterior nasal mucosa, comprises: (a) Aloe barbadensis leaf gel at about 0.0001 wt. % to about 0.01 wt. %, and sodium hyaluronate at about 0.0001 wt. % to about 0.01 wt. %; (b) a thickener; (c) an acid; (d) an alkaline agent; and (e) water, wherein each wt. % is based on the total weight of the composition. In various embodiments, this base composition may optionally comprise a surfactant, an amino acid, at least one inhalant, a salt, a fragrance, a preservative, a drug active, and/or a sweetener. In various aspects, the MW of the sodium hyaluronate is about 1×10⁶ to about 1.5×10⁶ Daltons.

In various embodiments, a composition in accordance with the present disclosure, useful for cleansing the nose, clearing the nose, imparting a cooling sensation in the nostrils, moisturizing nasal passages, soothing a dry nose, or ameliorating dehydration of the anterior nasal mucosa, comprises: (a) Aloe barbadensis leaf gel at about 0.0001 wt. % to about 0.01 wt. %, and sodium hyaluronate at about 0.0001 wt. % to about 0.01 wt. %; (b) a thickener at about 0.001 wt. % to about 10 wt. %; (c) an acid at about 0.01 wt. % to about 10 wt. %; (d) an alkaline agent at about 0.01 wt. % to about 10 wt. %; and (e) water, wherein each wt. % is based on the total weight of the composition. In various embodiments, this base composition may optionally comprise a surfactant, an amino acid, at least one inhalant, a salt, a fragrance, a preservative, a drug active, and/or a sweetener. In various aspects, the MW of the sodium hyaluronate is about 1×10⁶ to about 1.5×10⁶ Daltons.

In various embodiments, a composition in accordance with the present disclosure, useful for cleansing the nose, clearing the nose, imparting a cooling sensation in the nostrils, moisturizing nasal passages, soothing a dry nose, or ameliorating dehydration of the anterior nasal mucosa, comprises: (a) Aloe barbadensis leaf gel at about 0.0001 wt. % to about 0.01 wt. %, and sodium hyaluronate at about 0.0001 wt. % to about 0.01 wt. %; (b) a thickener; (c) a surfactant; (d) an acid; (e) an alkaline agent; and (f) water, wherein each wt. % is based on the total weight of the composition. In various embodiments, this base composition may optionally comprise an amino acid, at least one inhalant, a salt, a fragrance, a preservative, a drug active, and/or a sweetener. In various aspects, the MW of the sodium hyaluronate is about 1'10⁶ to about 1.5×10⁶ Daltons.

In various embodiments, a composition in accordance with the present disclosure, useful for cleansing the nose, clearing the nose, imparting a cooling sensation in the nostrils, moisturizing nasal passages, soothing a dry nose, or ameliorating dehydration of the anterior nasal mucosa, comprises: (a) Aloe barbadensis leaf gel at about 0.0001 wt. % to about 0.01 wt. %, and sodium hyaluronate at about 0.0001 wt. % to about 0.01 wt. %; (b) a thickener at about 0.001 wt. % to about 10 wt. %; (c) a surfactant at about 0.001 wt. % to about 10 wt. %; (d) an acid at about 0.01 wt. % to about 10 wt. %; (e) an alkaline agent at about 0.01 wt. % to about 10 wt. %; and (f) water, wherein each wt. % is based on the total weight of the composition. In various embodiments, this base composition may optionally comprise an amino acid, at least one inhalant, a salt, a fragrance, a preservative, a drug active, and/or a sweetener. In various aspects, the MW of the sodium hyaluronate is about 1×10⁶ to about 1.5×10⁶ Daltons.

In various embodiments, a composition in accordance with the present disclosure, useful for cleansing the nose, clearing the nose, imparting a cooling sensation in the nostrils, moisturizing nasal passages, soothing a dry nose, or ameliorating dehydration of the anterior nasal mucosa, comprises: (a) Aloe barbadensis leaf gel at about 0.0001 wt. % to about 0.01 wt. %, and sodium hyaluronate at about 0.0001 wt. % to about 0.01 wt. %; (b) a thickener; (c) a surfactant; (d) an acid; (e) an alkaline agent; (f) at least one inhalant; and (g) water, wherein each wt. % is based on the total weight of the composition. In various embodiments, this base composition may optionally comprise an amino acid, a salt, a fragrance, a preservative, a drug active, and/or a sweetener. In various aspects, the MW of the sodium hyaluronate is about 1×10⁶ to about 1.5×10⁶ Daltons.

In various embodiments, a composition in accordance with the present disclosure, useful for cleansing the nose, clearing the nose, imparting a cooling sensation in the nostrils, moisturizing nasal passages, soothing a dry nose, or ameliorating dehydration of the anterior nasal mucosa, comprises: (a) Aloe barbadensis leaf gel at about 0.0001 wt. % to about 0.01 wt. %, and sodium hyaluronate at about 0.0001 wt. % to about 0.01 wt. %; (b) a thickener at about 0.001 wt. % to about 10 wt. %; (c) a surfactant at about 0.001 wt. % to about 10 wt. %; (d) an acid at about 0.01 wt. % to about 10 wt. %; (e) an alkaline agent at about 0.01 wt. % to about 10 wt. %; (f) at least one inhalant at a total of about 0.001 wt. % to about 1 wt. %; and (g) water, wherein each wt. % is based on the total weight of the composition. In various embodiments, this base composition may optionally comprise an amino acid, a salt, a fragrance, a preservative, a drug active, and/or a sweetener. In various aspects, the MW of the sodium hyaluronate is about 1×10⁶ to about 1.5×10⁶ Daltons.

In various embodiments, a composition in accordance with the present disclosure, useful for cleansing the nose, clearing the nose, imparting a cooling sensation in the nostrils, moisturizing nasal passages, soothing a dry nose, or ameliorating dehydration of the anterior nasal mucosa, comprises: (a) Aloe barbadensis leaf gel at about 0.0001 wt. % to about 0.01 wt. %, and sodium hyaluronate at about 0.0001 wt. % to about 0.01 wt. %; (b) a thickener; (c) a surfactant; (d) an acid; (e) an alkaline agent; (f) at least one inhalant; (g) an amino acid; and (h) water, wherein each wt. % is based on the total weight of the composition. In various embodiments, this base composition may optionally comprise a salt, a fragrance, a preservative, a drug active, and/or a sweetener. In various aspects, the MW of the sodium hyaluronate is about 1×10⁶ to about 1.5×10⁶ Daltons.

In various embodiments, a composition in accordance with the present disclosure, useful for cleansing the nose, clearing the nose, imparting a cooling sensation in the nostrils, moisturizing nasal passages, soothing a dry nose, or ameliorating dehydration of the anterior nasal mucosa, comprises: (a) Aloe barbadensis leaf gel at about 0.0001 wt. % to about 0.01 wt. %, and sodium hyaluronate at about 0.0001 wt. % to about 0.01 wt. %; (b) a thickener at about 0.001 wt. % to about 10 wt. %; (c) a surfactant at about 0.001 wt. % to about 10 wt. %; (d) an acid at about 0.01 wt. % to about 10 wt. %; (e) an alkaline agent at about 0.01 wt. % to about 10 wt. %; (f) at least one inhalant at a total of about 0.001 wt. % to about 1 wt. %; (g) at least one amino acid at a total of about 0.01 wt. % to about 10 wt. %; and (h) water, wherein each wt. % is based on the total weight of the composition. In various embodiments, this base composition may optionally comprise a salt, a fragrance, a preservative, a drug active, and/or a sweetener. In various aspects, the MW of the sodium hyaluronate is about 1×10⁶ to about 1.5×10⁶ Daltons.

In various embodiments, a composition in accordance with the present disclosure, useful for cleansing the nose, clearing the nose, imparting a cooling sensation in the nostrils, moisturizing nasal passages, soothing a dry nose, or ameliorating dehydration of the anterior nasal mucosa, comprises: (a) Aloe barbadensis leaf gel at about 0.0001 wt. % to about 0.01 wt. %, and sodium hyaluronate at about 0.0001 wt. % to about 0.01 wt. %; (b) a thickener at about 0.001 wt. % to about 10 wt. %; (c) a surfactant at about 0.001 wt. % to about 10 wt. %; (d) an acid at about 0.01 wt. % to about 10 wt. %; (e) an alkaline agent at about 0.01 wt. % to about 10 wt. %; (f) at least one inhalant at a total of about 0.001 wt. % to about 1 wt. %; (g) optionally at least one amino acid at a total of about 0.01 wt. % to about 10 wt. %; (h) optionally at least one salt at about 0.01 wt. % to about 1 wt. %; (i) optionally at least one fragrance at about 0.001 wt. % to about 1 wt. %; (j) optionally at least one sweetener at about 0.001 wt. % to about 1 wt. %; (k) optionally at least one of a preservative, uv inhibitor and antioxidant at a total of about 0.001 wt. % to about 1 wt. %; (l) optionally a non-aqueous solvent or vehicle at about 0.001 wt. % to about 99 wt. %; and (m) remainder water, wherein each wt. % is based on the total weight of the composition. In various embodiments, the composition further comprises a drug active. In certain examples, the drug active comprises a decongestant or an antihistamine, or a mixture thereof. In various aspects, the MW of the sodium hyaluronate is about 1×10⁶ to about 1.5×10⁶ Daltons.

In various embodiments, a composition in accordance with the present disclosure, useful for cleansing the nose, clearing the nose, imparting a cooling sensation in the nostrils, moisturizing nasal passages, soothing a dry nose, or ameliorating dehydration of the anterior nasal mucosa, comprises: (a) Aloe barbadensis leaf gel at about 0.0001 wt. % to about 0.01 wt. %, and sodium hyaluronate at about 0.0001 wt. % to about 0.01 wt. %; (b) a cellulosic thickener at about 0.001 wt. % to about 10 wt. %; (c) a surfactant at about 0.001 wt. % to about 10 wt. %; (d) an acid at about 0.01 wt. % to about 10 wt. %; (e) an alkaline agent at about 0.01 wt. % to about 10 wt. %; (f) at least one inhalant at a total of about 0.001 wt. % to about 1 wt. %; (g) optionally at least one amino acid at a total of about 0.01 wt. % to about 10 wt. %; (h) optionally at least one salt at about 0.01 wt. % to about 1 wt. %; (i) optionally at least one fragrance at about 0.001 wt. % to about 1 wt. %; (j) optionally at least one sweetener at about 0.001 wt. % to about 1 wt. %; (k) optionally at least one of a preservative, uv inhibitor and antioxidant at a total of about 0.001 wt. % to about 1 wt. %; (l) optionally a non-aqueous solvent or vehicle at about 0.001 wt. % to about 99 wt. %; and (m) remainder water, wherein each wt. % is based on the total weight of the composition. In various embodiments, the composition further comprises a drug active. In certain examples, the drug active comprises a decongestant or an antihistamine, or a mixture thereof. In various aspects, the MW of the sodium hyaluronate is about 1×10⁶ to about 1.5'10⁶ Daltons.

In various embodiments, a composition in accordance with the present disclosure, useful for cleansing the nose, clearing the nose, imparting a cooling sensation in the nostrils, moisturizing nasal passages, soothing a dry nose, or ameliorating dehydration of the anterior nasal mucosa, comprises: (a) Aloe barbadensis leaf gel at about 0.0001 wt. % to about 0.01 wt. %, and sodium hyaluronate at about 0.0001 wt. % to about 0.01 wt. %; (b) a cellulosic thickener at about 0.001 wt. % to about 10 wt. %; (c) a nonionic surfactant at about 0.001 wt. % to about 10 wt. %; (d) an organic acid at about 0.01 wt. % to about 10 wt. %; (e) at least one of a carbonate, sulfate, and phosphate at a total of about 0.01 wt. % to about 10 wt. %; (f) at least one inhalant at a total of about 0.001 wt. % to about 1 wt. %; (g) optionally at least one amino acid at a total of about 0.01 wt. % to about 10 wt. %; (h) optionally at least one salt at a total of about 0.01 wt. % to about 1 wt. %; (i) optionally at least one fragrance at a total of about 0.001 wt. % to about 1 wt. %; (j) optionally at least one sweetener at about 0.001 wt. % to about 1 wt. %; (k) optionally at least one of a preservative, uv inhibitor and antioxidant at a total of about 0.001 wt. % to about 1 wt. %; (l) optionally a non-aqueous solvent or vehicle at about 0.001 wt. % to about 99 wt. %; and (m) remainder water, wherein each wt. % is based on the total weight of the composition. In various embodiments, the composition further comprises a drug active. In certain examples, the drug active comprises a decongestant or an antihistamine, or a mixture thereof. In various aspects, the MW of the sodium hyaluronate is about 1×10⁶ to about 1.5×10⁶ Daltons.

In various embodiments, a composition in accordance with the present disclosure, useful for cleansing the nose, clearing the nose, imparting a cooling sensation in the nostrils, moisturizing nasal passages, soothing a dry nose, or ameliorating dehydration of the anterior nasal mucosa, comprises: (a) Aloe barbadensis leaf gel at about 0.0001 wt. % to about 0.01 wt. %, and sodium hyaluronate at about 0.0001 wt. % to about 0.01 wt. %; (b) a cellulosic thickener at about 0.001 wt. % to about 10 wt. %; (c) a surfactant at about 0.001 wt. % to about 10 wt. %; (d) an organic acid at about 0.01 wt. % to about 10 wt. %; (e) at least one of a carbonate, sulfate, and phosphate at a total of about 0.01 wt. % to about 10 wt. %; (f) at least one inhalant at a total of about 0.001 wt. % to about 1 wt. %; (g) optionally at least one amino acid at a total of about 0.01 wt. % to about 10 wt. %; (h) optionally at least one salt at a total of about 0.01 wt. % to about 1 wt. %; (i) optionally at least one fragrance at a total of about 0.001 wt. % to about 1 wt. %; (j) optionally at least one sweetener at a total of about 0.001 wt. % to about 1 wt. %; (k) optionally at least one of a preservative, uv inhibitor and antioxidant at a total of about 0.001 wt. % to about 1 wt. %; (l) optionally a non-aqueous solvent or vehicle at about 0.001 wt. % to about 99 wt. %; (m) remainder water, wherein each wt. % is based on the total weight of the composition. In various embodiments, the composition further comprises a drug active. In certain examples, the drug active comprises a decongestant or an antihistamine, or a mixture thereof. In various aspects, the MW of the sodium hyaluronate is about 1×10⁶ to about 1.5×10⁶ Daltons.

In various embodiments, a composition in accordance with the present disclosure, useful for cleansing the nose, clearing the nose, imparting a cooling sensation in the nostrils, moisturizing nasal passages, soothing a dry nose, or ameliorating dehydration of the anterior nasal mucosa, comprises: (a) Aloe barbadensis leaf gel at about 0.0001 wt. % to about 0.01 wt. %, and sodium hyaluronate at about 0.0001 wt. % to about 0.01 wt. %; (b) a cellulosic thickener at about 0.5 wt. % to about 1.5 wt. %; (c) a nonionic surfactant at about 0.01 wt. % to about 1 wt. %; (d) an organic acid at about 0.1 wt. % to about 1 wt. %; (e) a mixture of monobasic and dibasic phosphate salts at a total of about 0.1 to about 2 wt. %; (f) a mixture of menthol, eucalyptol and eugenol at a total of about 0.1 wt. % to about 1 wt. %; (g) an α-amino acid at about 0.05 wt. % to about 0.5 wt. %; (h) sodium chloride at about 0.1 wt. % to about 1 wt. %; and (i) remainder water, wherein each wt. % is based on the total weight of the composition. In various embodiments, the composition further comprises 2-phenylethanol, and optionally any one of a preservative, uv inhibitor, antioxidant, and sweetener. In various embodiments, the cellulosic thickener comprises hypromellose. In various embodiments, said composition further includes a drug active. In certain examples, the drug active comprises a decongestant or an antihistamine, or a mixture thereof. In various aspects, the MW of the sodium hyaluronate is about 1×10⁶ to about 1.5×10⁶ Daltons.

In various embodiments, a liquid nasal spray, useful for cleansing the nose, clearing the nose, imparting a cooling sensation in the nostrils, moisturizing nasal passages, soothing a dry nose, or ameliorating dehydration of the anterior nasal mucosa, consists essentially of: (a) Aloe barbadensis leaf gel at about 0.0001 wt. % to about 0.01 wt. %, and sodium hyaluronate at about 0.0001 wt. % to about 0.01 wt. %; (b) a cellulosic thickener at about 0.5 wt. % to about 1.5 wt. %; (c) a polyethoxylated sorbitan fatty acid ester at about 0.01 wt. % to about 1 wt. %; (d) a di-carboxylic organic acid at about 0.1 wt. % to about 1 wt. %; (e) a mixture of monobasic and dibasic phosphate salts at a total of about 0.1 to about 2 wt. %; (f) a mixture of menthol, eucalyptol and eugenol at a total of about 0.1 wt. % to about 1 wt. %; (g) glycine at about 0.05 wt. % to about 0.5 wt. %; (h) sodium chloride at about 0.1 wt. % to about 1 wt. %; and (i) remainder water, wherein each wt. % is based on the total weight of the composition. In various embodiments, the composition further comprises 2-phenylethanol, and optionally any one of a preservative, uv inhibitor, antioxidant, and sweetener. In various embodiments, the cellulosic thickener comprises hypromellose. In various embodiments, the composition further includes a drug active. In certain examples, the drug active comprises a decongestant or an antihistamine, or a mixture thereof. In various aspects, the MW of the sodium hyaluronate is about 1×10⁶ to about 1.5×10⁶ Daltons.

In various embodiments, a liquid nasal spray, useful for cleansing the nose, clearing the nose, imparting a cooling sensation in the nostrils, moisturizing nasal passages, soothing a dry nose, or ameliorating dehydration of the anterior nasal mucosa, consists essentially of: (a) Aloe barbadensis leaf gel at about 0.001 wt. %; (b) sodium hyaluronate at about 0.001 wt. %; (c) a cellulosic thickener at about 1.0 wt. %; (d) a polyethoxylated sorbitan monooleate at about 0.08 wt. %; (e) succinic acid at about 0.385 wt. %; (f) NaH₂PO₄ at about 0.54 wt. %; (g) Na₂HPO₄ at about 0.65 wt. %; (h) menthol at about 0.06 wt. %; (i) eucalyptol at about 0.025 wt. %; (j) eugenol at about 0.002 wt. %; (k) glycine at about 0.1 wt. %; (l) sodium chloride at about 0.45 wt. %; (m) 2-phenylethanol at about 0.02 wt. %; (n) a sweetener at about 0.03 wt. %; (o) a preservative at about 0.02 wt. %; and (p) remainder water, wherein each wt. % is based on the total weight of the composition. In various aspects, the MW of the sodium hyaluronate is about 1×10⁶ to about 1.5×10⁶ Daltons.

In various embodiments, a method for cleansing the nose, clearing the nose, imparting a cooling sensation in the nostrils, moisturizing nasal passages, soothing a dry nose, or ameliorating dehydration of the anterior nasal mucosa in a human comprises nasal administration of a therapeutically effective amount of a pharmaceutical composition comprising: (a) at least one humectant selected from the group consisting of Aloe barbadensis leaf gel, hyaluronic acid, a hyaluronate salt, and mixtures thereof; (b) from about 0.001 wt. % to about 10 wt. % of a thickener; and (c) a pharmaceutically acceptable carrier comprising at least about 90 wt. % water, wherein each wt. % is based on the total weight of the composition. In various embodiments, the composition further comprises from about 0.01 wt. % to about 10 wt. % of an organic acid, and from about 0.01 wt. % to about 10 wt. % of an alkaline material. In various embodiments, the composition further comprises a total of from about 0.001 wt. % to about 1 wt. % of at least one inhalant. In various embodiments, the composition further comprises from about 0.01 wt. % to about 10 wt. % of an amino acid. In various embodiments, the composition further comprises from about 0.001 wt. % to about 10 wt. % of a surfactant. In various embodiments, the composition further comprises at least one drug active. In various embodiments, the therapeutically effective amount comprises from about 50 μL to about 250 μL of the composition. In various embodiments, the therapeutically effective amount comprises about 140 μL to about 150 μL of the composition.

In various embodiments, a method for cleansing the nose, clearing the nose, imparting a cooling sensation in the nostrils, moisturizing nasal passages, soothing a dry nose, or ameliorating dehydration of the anterior nasal mucosa in a human comprises nasal administration of a therapeutically effective amount of a pharmaceutical composition consisting essentially of: (a) from about 0.0002 wt. % to about 0.02 wt. % of a mixture of Aloe barbadensis leaf gel and a hyaluronate salt; (b) from about 0.5 wt. % to about 1.5 wt. % of a cellulosic thickener; (c) from about 0.01 wt. % to about 1 wt. % of a polyethoxylated sorbitan fatty acid ester; (d) from about 0.1 wt. % to about 1 wt. % of a di-carboxylic organic acid; (e) from about 0.1 to about 2 wt. % of a mixture of sodium monobasic and sodium dibasic phosphate salts; (f) from about 0.01 wt. % to about 1 wt. % of a mixture of menthol, eucalyptol and eugenol; (g) from about 0.05 wt. % to about 0.5 wt. % glycine; (h) from about 0.1 wt. % to about 1 wt. % sodium chloride; and (i) from about 90 wt. % to about 99 wt. % water, wherein each wt. % is based on the total weight of the composition. In various embodiments, the composition optionally includes 2-phenylethanol, any one of a preservative, uv inhibitor and antioxidant, and a sweetener. In various embodiments, the cellulosic thickener comprises hypromellose. In various embodiments, the composition further includes a drug active. In certain examples, the drug active comprises a decongestant or an antihistamine, or a mixture thereof. In various aspects, the MW of the hyaluronate salt is about 1×10⁶ to about 1.5×10⁶ Daltons. In various embodiments, the therapeutically effective amount comprises from about 50 μL to about 250 μL of the composition. In various embodiments, the therapeutically effective amount comprises about 140 μL to about 150 μL of the composition.

In various embodiments, a method for cleansing the nose, clearing the nose, imparting a cooling sensation in the nostrils, moisturizing nasal passages, soothing a dry nose, or ameliorating dehydration of the anterior nasal mucosa in a human comprises nasal administration of a therapeutically effective amount of a pharmaceutical composition comprising: (a) from about 0.0001 wt. % to about 0.01 wt. % Aloe barbadensis leaf gel; (b) from about 0.0001 wt. % to about 0.01 wt. % of a hyaluronate salt; (c) from about 0.001 wt. % to about 10 wt. % of a thickener; and (d) a pharmaceutically acceptable carrier comprising at least about 90 wt. % water, wherein each wt. % is based on the total weight of the composition. In various embodiments, the composition further comprises from about 0.01 wt. % to about 10 wt. % of an organic acid, and from about 0.01 wt. % to about 10 wt. % of an alkaline material. In various embodiments, the composition further comprises a total of from about 0.001 wt. % to about 1 wt. % of at least one inhalant. In various embodiments, the composition further comprises from about 0.01 wt. % to about 10 wt. % of an amino acid. In various embodiments, the composition further comprises from about 0.001 wt. % to about 10 wt. % of a surfactant. In various embodiments, the composition further comprises at least one drug active. In various aspects, the MW of the hyaluronate salt is about 1×10⁶ to about 1.5×10⁶ Daltons. In various embodiments, the therapeutically effective amount comprises from about 50 μL to about 250 μL of said composition. In various embodiments, the therapeutically effective amount comprises about 140 μL to about 150 μL of the composition.

In various embodiments, a method for cleansing the nose, clearing the nose, imparting a cooling sensation in the nostrils, moisturizing nasal passages, soothing a dry nose, or ameliorating dehydration of the anterior nasal mucosa in a human comprises nasal administration of a therapeutically effective amount of a pharmaceutical composition consisting essentially of: (a) from about 0.0001 wt. % to about 0.01 wt. % Aloe barbadensis leaf gel; (b) from about 0.0001 wt. % to about 0.01 wt. % of a hyaluronate salt; (c) from about 0.5 wt. % to about 1.5 wt. % of a cellulosic thickener; (d) from about 0.01 wt. % to about 1 wt. % of a polyethoxylated sorbitan fatty acid ester; (e) from about 0.1 wt. % to about 1 wt. % of a di-carboxylic organic acid; (f) from about 0.1 to about 2 wt. % of a mixture of sodium monobasic and sodium dibasic phosphate salts; (g) from about 0.01 wt. % to about 1 wt. % of a mixture of menthol, eucalyptol and eugenol; (h) from about 0.05 wt. % to about 0.5 wt. % glycine; (i) from about 0.1 wt. % to about 1 wt. % sodium chloride; and (j) from about 90 wt. % to about 99 wt. % water, wherein each wt. % is based on the total weight of the composition. In various embodiments, the composition optionally includes 2-phenylethanol, any one of a preservative, uv inhibitor and antioxidant, and a sweetener. In various embodiments, the cellulosic thickener is hypromellose. In various embodiments, the hyaluronate salt comprises sodium hyaluronate having a molecular weight of about 1.0×10⁶ to about 1.5×10⁶ Daltons. In various embodiments, the composition further includes a drug active. In various embodiments, said therapeutically effective amount comprises from about 50 μL to about 250 μL of said composition. In various embodiments, the therapeutically effective amount comprises about 140 μL to about 150 μL of the composition.

In-Home Use Test (IHUT)

Test Product and General Parameters:

The nasal relief composition of Table 3 was placed into a consumer IHUT in the product form of a pre-treated swab dispensing package as the “test product” in the IHUT. Each swab package contained 1 mL of the Table 3 composition. Participants were instructed to break open the swab container, remove the swab, nasally administer the liquid from the swab into one nostril, reinsert the swab into the package to allow additional composition to absorb into the swab tip, and nasally administer the liquid from the swab into the other nostril. For each nasal administration, about 140 μL of composition transferred from the saturated swab tip to the inside of the participant's nostril.

Participants in the IHUT were allowed to use the product at a frequency of their own choosing, since frequency of use was a variable of interest from the study. Participants primarily reported using the test product on a daily basis, either in the morning or before going to bed at night. Participants in the test appeared motivated to use the test product to help ease congestion and irritation as well as to clear excess mucus.

Criteria for Evaluation of Efficacy:

The following five criteria were evaluated by tallying feedback from the consumer participants in the IHUT: comfort using the product; perception of clean; perception of clear; perception of soothing; and perception of cooling. Of particular interest was whether the test product delivered on a marketing promise of “clearing, cleansing, and soothing” the nose. Since each of these sensory factors are abstract in that they relate to personal feelings and are not conceivably measurable as physiological effects in the nostril by any scientific means, the proof of efficacy comes from favorable consumer testing as statistically analyzed from user questionnaires.

Participants for the IHUT were screened on two criteria. Namely, people who had suffered from a cough, cold, allergy, flu or sinus condition in the past 12 months and have primary or shared responsibility for purchase of cough, cold, allergy or sinus medications in the household. Although participants were asked if they were currently suffering from these conditions, the existence of a current condition was not criteria for exclusion from the IHUT. The participants were segregated into three subgroups: General Population, which includes those participants not using any prescription or OTC nasal products and who are neutral in attitude or against use of same; CCAS sufferers (or referred to as CCAS users), which includes those participants who regularly use or are currently using a cough, cold, allergy or sinus product because of a past or current condition (as mentioned above regarding recruitment); and Nasal Acceptors, which are CCAS sufferers who currently use or are open to using a nasal product form. As mentioned, CCAS sufferers who happened to be currently using a nasal CCAS product were not excluded from the IHUT.

All statistical testing was at the 90% confidence interval. In any of the tables below, the letter(s) in CAPS in the table adjacent to a particular percentage indicates that the percentage having the lettering and the entry or entries in the column(s) indicated by the lettering are significantly different statistically. General Population subgroup is column A. Current CCAS Sufferer subgroup is column B. Nasal Acceptors subgroup is column C.

Results:

1. Comfort using Product

Consumer participants were asked, “how comfortable or uncomfortable was the experience of using the test product?” Table 4 sets forth the results from the questionnaires.

TABLE 4
Comfort Using Product

| | Subgroup | | |
|---|---|---|---|
| | General | Current CCAS | Nasal |
| | Population | Sufferers | Acceptors |
| Participants reporting | N = 191 | N = 92 | N = 129 |
| Very Comfortable | 60% | 69% AC | 65% A |
| Somewhat comfortable | 21% | 16% | 21% |
| Neither comfortable | 9% | 8% | 7% |
| nor uncomfortable | | | |
| Somewhat uncomfortable | 9% BC | 5% | 5% |
| Very uncomfortable | 2% | 1% | 2% |

2. Perception of Clean

Consumer participants were asked, “how clean did your nose feel after using the test product?” Table 5 sets forth the results from the questionnaires.

TABLE 5
Perception of Clean

| | Subgroup | | |
|---|---|---|---|
| | General | Current CCAS | Nasal |
| | Population | Sufferers | Acceptors |
| Participants reporting | N = 191 | N = 92 | N = 129 |
| Extremely clean | 32% | 33% A | 38% AB |
| Very clean | 41% | 43% A | 43% A |
| Somewhat clean | 16% C | 16% C | 11% |
| Slightly clean | 7% C | 4% | 4% |
| Not clean at all | 5% B | 3% | 4% |

3. Perception of Clear

Consumer participants were asked, “how clear did your nose feel after using the test product?” Table 6 sets forth the results from the questionnaires.

TABLE 6
Perception of Clear

| | Subgroup | | |
|---|---|---|---|
| | General | Current CCAS | Nasal |
| | Population | Sufferers | Acceptors |
| Participants reporting | N = 191 | N = 92 | N = 129 |
| Extremely clear | 35% | 40% A | 41% A |
| Very clear | 37% | 43% A | 41% A |
| Somewhat clear | 16% BC | 10% | 12% |
| Slightly clear | 4% C | 4% | 0% |
| Not clear at all | 8% BC | 4% | 6% |

4. Perception of Soothing

Consumer participants were asked, “how well did the test product soothe any irritation in your nose?” Table 7 sets forth the results from the questionnaires.

TABLE 7
Perception of Soothing

| | Subgroup | | |
|---|---|---|---|
| | General | Current CCAS | Nasal |
| | Population | Sufferers | Acceptors |
| Participants reporting | N = 191 | N = 92 | N = 129 |
| Extremely well | 32% | 44% AC | 38% A |
| Very well | 42% B | 37% | 45% AB |
| Somewhat well | 14% C | 13% | 11% |
| Slightly well | 6% C | 3% C | 1% |
| Not well at all | 6% B | 3% | 5% |

5. Perception of Cooling

Consumer participants were asked, “how long did the cooling sensation last after using the test product?” Table 8 sets forth the results from the questionnaires.

TABLE 8
Perception of Cooling

| | Subgroup | | |
|---|---|---|---|
| | General | Current CCAS | Nasal |
| | Population | Sufferers | Acceptors |
| Participants reporting | N = 191 | N = 92 | N = 129 |
| Less than 1 minute | 12% | 10% | 14% AB |
| A few minutes | 51% BC | 46% | 48% B |
| About 15 minutes | 19% | 25% AC | 20% |
| About 30 minutes | 10% C | 9% | 8% |
| About an hour | 3% | 2% | 3% |
| More than 1 hour | 5% | 8% A | 6% |

Table 9 sets forth the subgroup means, which questions whether or not the test product delivered consumer expectations.

TABLE 9
Subgroup Means

| | Subgroup | | |
|---|---|---|---|
| | General | Current CCAS | Nasal |
| | Population | Sufferers | Acceptors |
| Participants reporting | N = 191 | N = 92 | N = 129 |

PRODUCT DELIVERY

| | | | |
|---|---|---|---|
| Better than expected | 59.5% | 67.7% AC | 65.2% A |
| Same as expected | 27.2% C | 26.7% C | 24.3% |
| Worse than expected | 13.3% BC | 5.6% | 10.5% B |

As indicated from the statistically analyzed data in Tables 4-9, the test product, (the composition of Table 3 in a pre-treated swab delivery package), delivered on consumer expectations. A statistically significant number of consumers found the test product to clear the nose, cleanse the nose, and soothe the nose. Consumers primarily reported the cooling sensation in the nose to last a few minutes. Given the data, it is evident that the pharmaceutical composition of Table 2 provided the benefits of cleansing the nose, clearing the nose, imparting a cooling sensation in the nostrils, moisturizing nasal passages, soothing a dry nose, and ameliorating dehydration of the anterior nasal mucosa in that a statistically significant number of participants noted relief

In the detailed description, references to “various embodiments”, “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to ‘at least one of A, B, and C’ or ‘at least one of A, B, or C’ is used in the claims or specification, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C.

All structural, chemical, and functional equivalents to the elements of the above-described various embodiments that are known to those of ordinary skill in the art are expressly incorporated herein by reference and are intended to be encompassed by the present claims. Moreover, it is not necessary for an apparatus or component of an apparatus, or method in using an apparatus to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.” As used herein, the terms “comprise,” “comprises,” “comprising,” or any other variation thereof, are intended to cover a non-exclusive inclusion, such that a chemical, chemical composition, process, method, article, or apparatus that comprises a list of elements does not include only those elements but may include other elements not expressly listed or inherent to such chemical, chemical composition, process, method, article, or apparatus.

What is claimed is:

1. A method for cleansing the nose, clearing the nose, imparting a cooling sensation in the nostrils, moisturizing nasal passages, soothing a dry nose, or ameliorating dehydration of the anterior nasal mucosa, the method comprising: nasally administering to a human in need of relief thereof a therapeutically effective amount of a composition comprising: (a) at least one humectant totaling about 0.0002 wt. % to about 0.02 wt. %; (b) a thickener at from about 0.001 wt. % to about 10 wt. %; and (c) a pharmaceutically acceptable carrier comprising at least about 90 wt. % water.

2. The method according to claim 1, wherein the composition further comprises from about 0.01 wt. % to about 10 wt. % of an organic acid, and from about 0.01 wt. % to about 10 wt. % of an alkaline material.

3. The method according to claim 1, wherein the composition further comprises at least one inhalant totaling from about 0.001 wt. % to about 1 wt. %.

4. The method according to claim 1, wherein the composition further comprises from about 0.01 wt. % to about 10 wt. % of an amino acid.

5. The method according to claim 1, wherein the composition further comprises from about 0.001 wt. % to about 10 wt. % of a surfactant.

6. The method according to claim 1, wherein the composition further comprises at least one drug active.

7. The method according to claim 1, wherein the therapeutically effective amount comprises from about 50 μL to about 250 μL of said composition.

8. A method for cleansing the nose, clearing the nose, imparting a cooling sensation in the nostrils, moisturizing nasal passages, soothing a dry nose, or ameliorating dehydration of the anterior nasal mucosa, the method comprising: nasally administering to a human in need of relief thereof a composition comprising: (a) from about 0.0001 wt. % to about 0.01 wt. % of Aloe barbadensis leaf gel; (b) from about 0.0001 wt. % to about 0.01 wt. % of sodium hyaluronate; (c) from about 0.5 wt. % to about 1.5 wt. % of a cellulosic thickener; (d) from about 0.01 wt. % to about 1 wt. % of a surfactant; (e) from about 0.1 wt. % to about 1 wt. % of an organic acid; (f) from about 0.1 to about 2 wt. % of at least one alkaline material; (g) from about 0.01 wt. % to about 1 wt. % of an inhalant selected from the group consisting of eucalyptol, eugenol, menthol, camphor, and mixtures thereof; (h) from about 0.05 wt. % to about 0.5 wt. % of an amino acid;
(i) from about 0.1 wt. % to about 1 wt. % sodium chloride; and (j) from about 90 wt. % to about 99 wt. % water, wherein each wt. % is based on the total weight of the composition.

9. The method according to claim 8, wherein the cellulosic thickener is hypromellose.

10. The method according to claim 8, wherein the surfactant is polysorbate 80.

11. The method according to claim 8, wherein the organic acid is succinic acid.

12. The method according to claim 8, wherein the at least one alkaline material is a mixture of sodium monobasic and sodium dibasic phosphate salts.

13. The method according to claim 8, wherein the inhalant is a mixture of eucalyptol, eugenol and menthol.

14. The method according to claim 8, wherein the amino acid is chosen from the group consisting of glycine, L-lysine, and mixtures thereof.

15. The method according to claim 8, wherein the sodium hyaluronate has a molecular weight of about 1.0×10⁶ to 1.5×10⁶ Daltons.

16. The method according to claim 8, wherein the composition further comprises 2-phenylethanol at a level of about 0.001 wt. % to about 1 wt. %, based on the total weight of the composition.

17. The method according to claim 8, wherein the composition further comprises at least one drug active selected from the group consisting of decongestants, antihistamines and mixtures thereof.

18. The method according to claim 8, wherein the therapeutically effective amount comprises from about 50 μL to about 250 μL of said composition.

19. A method for cleansing the nose, clearing the nose, imparting a cooling sensation in the nostrils, moisturizing nasal passages, soothing a dry nose, or ameliorating dehydration of the anterior nasal mucosa, the method comprising: nasally administering to a human in need of relief thereof a composition comprising: (a) Aloe barbadensis leaf gel at about 0.001 wt. %; (b) sodium hyaluronate at about 0.001 wt. %; (c) a cellulosic thickener at about 1.0 wt. %; (d) a0 polyethoxylated sorbitan monooleate at about 0.08 wt. %; (e) succinic acid at about 0.385 wt. %; (f) NaH₂PO₄ at about 0.54 wt. %; (g) Na₂HPO₄ at about 0.65 wt. %; (h) menthol at about 0.06 wt. %; (i) eucalyptol at about 0.025 wt. %; (j) eugenol at about 0.002 wt. %; (k) glycine at about 0.1 wt. %; (l) sodium chloride at about 0.45 wt. %; (m) 2-phenylethanol at about 0.02 wt. %; (n) a sweetener at about 0.03 wt. %; (o) a preservative at about 0.02 wt. %; and (p) remainder water, wherein each wt. % is based on the total weight of the composition.

20. The method according to claim 19, wherein the sodium hyaluronate has a molecular weight of about 1×10⁶ to about 1.5×10⁶ Daltons.

21. The method according to claim 19, wherein the therapeutically effective amount comprises from about 50 μL to about 250 μL of said composition.

22. The method according to claim 19, wherein the cellulosic thickener comprises hypromellose.

23. A pharmaceutical composition comprising: (a) at least one humectant totaling about 0.0002 wt. % to about 0.02 wt. %; (b) a thickener at from about 0.001 wt. % to about 10 wt. %; and (c) a pharmaceutically acceptable carrier comprising at least about 90 wt. % water.

24. The composition according to claim 23, wherein the composition further comprises from about 0.01 wt. % to about 10 wt. % of an organic acid, and from about 0.01 wt. % to about 10 wt. % of an alkaline material.

25. The composition according to claim 23, wherein the composition further comprises at least one inhalant totaling from about 0.001 wt. % to about 1 wt. %.

26. The composition according to claim 23, wherein the composition further comprises from about 0.01 wt. % to about 10 wt. % of an amino acid.

27. The composition according to claim 23, wherein the composition further comprises from about 0.001 wt. % to about 10 wt. % of a surfactant.

28. The composition according to claim 23, wherein the composition further comprises at least one drug active.

29. A pharmaceutical composition comprising: (a) from about 0.0001 wt. % to about 0.01 wt. % of Aloe barbadensis leaf gel; (b) from about 0.0001 wt. % to about 0.01 wt. % of sodium hyaluronate; (c) from about 0.5 wt. % to about 1.5 wt. % of a cellulosic thickener; (d) from about 0.01 wt. % to about 1 wt. % of a surfactant; (e) from about 0.1 wt. % to about 1 wt. % of an organic acid; (f) from about 0.1 to about 2 wt. % of at least one alkaline material; (g) from about 0.01 wt. % to about 1 wt. % of an inhalant selected from the group consisting of eucalyptol, eugenol, menthol, camphor, and mixtures thereof; (h) from about 0.05 wt. % to about 0.5 wt. % of an amino acid; (i) from about 0.1 wt. % to about 1 wt. % sodium chloride; and (j) from about 90 wt. % to about 99 wt. % water, wherein each wt. % is based on the total weight of the composition.

30. The composition according to claim 29, wherein the cellulosic thickener is hypromellose.

31. The composition according to claim 29, wherein the surfactant is polysorbate 80.

32. The composition according to claim 29, wherein the organic acid is succinic acid.

33. The composition according to claim 29, wherein the at least one alkaline material is a mixture of sodium monobasic and sodium dibasic phosphate salts.

34. The composition according to claim 29, wherein the inhalant is a mixture of eucalyptol, eugenol and menthol.

35. The composition according to claim 29, wherein the amino acid is chosen from the group consisting of glycine, L-lysine, and mixtures thereof.

36. The composition according to claim 29, wherein the sodium hyaluronate has a molecular weight of about 1.0×10⁶ to 1.5×10⁶ Daltons.

37. The composition according to claim 29, wherein the composition further comprises 2-phenylethanol at a level of about 0.001 wt. % to about 1 wt. %, based on the total weight of the composition.

38. The composition according to claim 29, wherein the composition further comprises at least one drug active selected from the group consisting of decongestants, antihistamines and mixtures thereof.

39. A pharmaceutical composition consisting essentially of: (a) Aloe barbadensis leaf gel at about 0.001 wt. %; (b) sodium hyaluronate at about 0.001 wt. %; (c) hypromellose at about 1.0 wt. %; (d) a polyethoxylated sorbitan monooleate at about 0.08 wt. %; (e) succinic acid at about 0.385 wt. %; (f) NaH₂PO₄ at about 0.54 wt. %; (g) Na₂HPO₄ at about 0.65 wt. %; (h) menthol at about 0.06 wt. %; (i) eucalyptol at about 0.025 wt. %; (j) eugenol at about 0.002 wt. %; (k) glycine at about 0.1 wt. %; (l) sodium chloride at about 0.45 wt. %; (m) 2-phenylethanol at about 0.02 wt. %; (n) a sweetener at about 0.03 wt. %; (o) a preservative at about 0.02 wt. %; and (p) remainder water, wherein each wt. % is based on the total weight of the composition.

40. The composition according to claim 39, wherein the sodium hyaluronate has a molecular weight of about 1×10⁶ to about 1.5×10⁶ Daltons.

41. A pre-treated swab dispensing system comprising: a pre-treated swab within a sealed reservoir tube, the reservoir tube containing a quantity of a composition of Table 2, wherein the pre-treated swab comprises an absorbent tip saturated with the composition.

42. The pre-treated swab dispensing system of claim 41, wherein the composition consists essentially of the composition in Table 3.

43. The pre-treated swab dispensing system of claim 42, wherein the quantity of composition in the reservoir tube is about 1 mL per reservoir tube.

44. A method of nasally administrating a pharmaceutical composition, the method comprising:
grasping and snapping open a pre-treated swab dispensing system comprising a pre-treated swab inside a sealed reservoir tube containing the pharmaceutical composition, the swab comprising an absorbent tip saturated with the pharmaceutical composition;
pulling out the pre-treated swab from the reservoir tube; and
nasally administering the pharmaceutical composition from the swab tip into a first nostril.

45. The method of claim 44, wherein the pharmaceutical composition comprising a composition of Table 2.

46. The method of claim 44, wherein the pharmaceutical composition comprises the composition of Table 3.

47. The method of claim 44, further comprising the step of reinserting the swab into the reservoir tube to re-saturate the swab tip with additional pharmaceutical composition; and
nasally administering the additional pharmaceutical composition into a second nostril.

48. The method of claim 44, wherein the sealed reservoir tube contains approximately 1 mL of the pharmaceutical composition of Table 3.

49. The method of claim 47, wherein nasally administering the composition comprises nasally administering from about 50 μL to about 250 μL of the composition into each nostril.

50. The method of claim 49, wherein nasally administering the composition comprises nasally administering about 140 μL of the composition into each nostril.
